(19) United States
(12) Patent Application Publication
(10) Pub. No.: US 2023/0001049 A1
(43) Pub. Date: Jan. 5, 2023

(54) Scaffold Using Adipose Tissue-Derived Extracellular Matrix and Method for Producing Same

(71) Applicant: L&C Bio Co., Ltd., Gyeonggi-do (KR)

(72) Inventors: Sang Chul Kim, Seoul (KR); Jang Il Kim, Seoul (KR); Hyung Gu Kim, Seoul (KR); Whan Chul Lee, Seoul (KR)

(73) Assignee: L&C Bio Co., Ltd., Gyeonggi-do (KR)

(21) Appl. No.: 17/781,879

(22) PCT Filed: Dec. 9, 2019
(86) PCT No.: PCT/KR2019/017269

§ 371 Date: Jun. 2, 2022

(51) Int. Cl. A61L 27/36

(52) U.S. Cl. CPC A61L 27/3604; A61L 27/3687; A61L 27/3691

(57) ABSTRACT

The present invention relates to an allogeneic and heterologous adipose tissue-derived extracellular matrix scaffold, and a method for producing the same.

An adipose tissue-derived extracellular matrix scaffold according to the present invention has a composition similar to the human body, a large surface area, and an interconnected porous structure, and thus has high cell affinity and allows cells to survive for long periods.

### TECHNICAL FIELD

The present invention relates to an extracellular matrix scaffold derived from allogeneic or heterologous adipose tissue and a method of producing the same. More particularly, the present invention relates to an extracellular matrix scaffold derived from allogeneic or heterologous adipose tissue, which has a porous structure having components similar to the human body, a wide surface area, and interconnected pores, so it can have high cell affinity and allow cells to survive for a long time, and a method of producing the same.

### TECHNICAL FIELD

Regenerative medicine aims to replace or regenerate human cells, tissue, and organs. Physical trauma that causes tissue damage and functional loss and the emergence of new diseases caused by the advancement of society provide the inevitable motivation for more rapid development of the field of regenerative medicine.

Medical materials used in the field of regenerative medicine have to be deliberately selected according to the type of tissue or organ to be applied to, the type of disease or trauma, and a patient's medical history. Typically, materials most frequently selected for research are heterologous collagen and gelatin, microorganism-derived hyaluronic acid, chitosan, a vegetable cellulose-based polymer, and vegetable alginate. In addition, allogeneic materials that can be obtained from human corpses are also attracting attention as effective biomaterials that can be safely used in the field of regenerative medicine.

Safety, effectiveness, and economic/industrial interests of, particularly, adipose tissue among biomaterials are growing at home and abroad. Adipose tissue is a loose connective tissue composed of adipocytes, preadipocytes, fibroblasts, vascular endothelial cells, and various immune cells. Adipose tissue contains an extracellular matrix such as collagen, elastin, laminin, fibronectin, or glycosaminoglycan. An extracellular matrix not only helps the support and proliferation of cells in tissue in the body but also maintains the composition of tissue by binding with cells, resulting in helping in the recovery of a damaged area of the body.

The allogeneic or heterologous adipose tissue-derived extracellular matrix is being studied in terms of various scaffolds for the replacement and reinforcement of damaged human tissue, and cell culture. Recently, in preclinical trials, it has been reported that adipose tissue-derived extracellular matrix scaffolds affect the repair of defective tissue. In addition, it has been reported that the adipose tissue-derived extracellular matrix scaffolds affect the growth of cells, which is due to their porous structure and components.

However, these allogeneic or heterologous adipose tissue-derived extracellular matrix scaffolds are generally produced using a combination of a surfactant and an enzyme. However, this conventional production method collapses the porous structure of the extracellular matrix and inhibits cell growth, which is the purpose of a scaffold. In addition, this method has a disadvantage in that a long-term production process is required.

### RELATED ART DOCUMENTS

### Patent Documents

1. Korean Patent No. 10-0771058

2. Korean Patent No. 10-1628821

### Non-Patent Documents

1. Combining decellularized human adipose tissue extracellular matrix and adipose-derived stem cells for adipose tissue engineering, Acta Biomaterialia 2013, 8921-31

2. Biocompatibility of injectable hydrogel from decellularized human adipose tissue in vitro and in vivo, Journal of Biomedical Materials Research Part B, 2018, 1684-1694

3. The use of decellularized adipose tissue to provide an inductive microenvironment for the adipogenic differentiation of human adipose-derived stem cells, Biomaterials, 2010, 4715-24

4. Simulation of tissue differentiation in a scaffold as a function of porosity, Young's modulus and dissolution rate: Application of mechanobiological models in tissue engineering, Biomaterials, 207, 5544-5554

### DISCLOSURE

### Technical Problem

Therefore, the present invention is directed to providing an adipose tissue-derived extracellular matrix scaffold, which has a porous structure having components similar to the human body, a wide surface area, and interconnected pores, so it can have high cell affinity and allow cells to survive for a long time and a method of producing the same.

More specifically, the present invention is directed to providing an adipose tissue-derived extracellular matrix scaffold, which can have low toxicity and high cell affinity, induce autologous tissue formation, reduce a production period, and realize low production costs and a production method thereof.

### Technical Solution

The present invention provides a method of producing an adipose tissue-derived extracellular matrix scaffold, which comprises: a delipidation step of removing a lipid component from adipose tissue;

a decellularization step of removing cells from the lipid component-removed adipose tissue; and

a lyophilization step of lyophilizing the cell-removed adipose tissue,

wherein the decellularization step is performed using a basic solution.

In addition, the present invention provides an adipose tissue-derived extracellular matrix scaffold produced by the above-described production method.

### Advantageous Effects

The present invention provides a novel production method for manufacturing an adipose tissue-derived extracellular matrix scaffold. Conventionally, the manufacturing of the adipose tissue-derived extracellular matrix scaffold takes approximately 7 to 10 days, but the period of the production method according to the present invention can be shortened to 3 days or less.

In addition, during decellularization, a basic solution can be used to induce the porous structure to be well maintained in the extracellular matrix and to contain the active ingredient of adipose tissue. In addition, according to this, an extracellular matrix scaffold in which cells can survive for a long time due to improved cell affinity can be provided.

### DESCRIPTION OF DRAWINGS

FIG. 1 is an image of various forms of extracellular matrix scaffolds according to an embodiment of the present invention.

FIG. 2 is a set of images showing Oil Red O staining for confirming the residual amount of fat in an extracellular matrix scaffold according to an embodiment of the present invention.

FIG. 3 is a set of images of DAPI staining and a graph quantifying DNA to confirm the residual amount of cells in an extracellular matrix scaffold according to one embodiment of the present invention.

FIG. 4 is a set of scanning electron microscope images for analyzing the structures of extracellular matrix scaffolds according to an embodiment of the present invention.

FIG. 5 is a set of images obtained by a live/dead cell viability assay kit and a graph quantifying a cell count to analyze cell growth in extracellular matrix scaffolds according to one embodiment of the present invention.

### MODES OF THE INVENTION

The present invention provides a method of producing an adipose tissue-derived extracellular matrix scaffold, which includes: a delipidation step of removing a lipid component from adipose tissue;

a decellularization step of removing cells from the lipid component-removed adipose tissue; and

a lyophilization step of lyophilizing the cell-removed adipose tissue.

In one embodiment of the present invention, it was confirmed that, by preparing an adipose tissue-derived extracellular matrix scaffold according to the steps of the present invention, a scaffold that has a uniform porous structure is produced, compared to a conventional extracellular matrix scaffold produced using a surfactant and an enzyme as a comparative example, and the survival and growth of cells in the scaffold are excellent.

Hereinafter, the method of producing an adipose tissue-derived extracellular matrix scaffold will be described in further detail.

The method of producing an adipose tissue-derived extracellular matrix scaffold (hereinafter, referred to as extracellular matrix scaffold) according to the present invention includes a delipidation step; a decellularization step; and a lyophilization step.

In one embodiment, the extracellular matrix (ECM) refers to a complex assembly of biopolymers filling the space in tissue or outside a cell. The extracellular matrix may have different components according to the type of cell or the degree of cell differentiation, and consist of a fibrous protein such as collagen or elastin, a complex protein such as proteoglycan or glycosaminoglycan, and a cell-adhesion glycoprotein such as fibronectin or laminin.

In one embodiment, the adipose tissue may be an allogeneic or heterologous adipose tissue. The “allogeneic” means human-derived, and the “heterologous” means being derived from animals other than a human, that is, mammals such as a pig, a cow or a horse, and fish.

That is, in the present invention, the extracellular matrix may be produced according to the production method of the present invention using allogeneic or heterologous adipose tissue.

In the present invention, before the delipidation step, a washing step may be performed. In the washing step, the adipose tissue may be washed with sterile distilled water. Through this step, impurities in the adipose tissue may be removed.

In the present invention, the delipidation step is a step of removing a lipid component from adipose tissue.

In one embodiment, delipidation means the removal of a lipid component from tissue.

In one embodiment, the removal of a lipid component may be performed by physical treatment or chemical treatment, or a combination of the physical and chemical treatments. When the physical and chemical treatments are performed in combination, the order of performance is not limited.

In one embodiment, the type of physical treatment is not particularly limited, and the physical treatment may be performed through pulverization. The pulverization may be performed using a pulverizing means that is known in the art, such as a mixer, a homogenizer, a freezing grinder, an ultrasonic grinder, a hand blender, or a plunger mill.

In pulverization, the particle diameter of the pulverized product, that is, pulverized adipose tissue may be 0.01 to 1 mm.

In one embodiment, the type of chemical treatment is not particularly limited, and the chemical treatment may be performed using a delipidation solution. The delipidation solution may include a polar solvent, a non-polar solvent, or a mixed solvent thereof. The polar solvent may be water, alcohol, or a mixed solvent thereof, and the alcohol, methanol, ethanol or isopropyl alcohol may be used. The non-polar solvent may be heptane, octane, or a mixed solution thereof. Specifically, in the present invention, as a delipidation solution, a mixed solution of isopropyl alcohol and hexane may be used. Here, a mixing ratio of isopropyl alcohol and hexane may be 40:60 to 60:40.

The treatment time of the delipidation solution may be 4 to 30 hours or 10 to 20 hours.

In one embodiment, the delipidation step may be performed by sequentially applying physical treatment and chemical treatment. The lipid component may be first eliminated from the adipose tissue through physical treatment, and the lipid component that is not eliminated by the physical treatment may be removed by chemical treatment.

In the present invention, the decellularization step may be a step of removing cells from the adipose tissue from which the lipid component is removed in the delipidation step.

In one embodiment, decellularization means the removal of other cell components excluding the extracellular matrix from the tissue, for example, the nucleus, the cell membrane, nucleic acids, and the like.

In one embodiment, the decellularization may be performed using a basic solution, and as the basic solution, one or more selected from the group consisting of sodium hydroxide, potassium hydroxide, ammonium hydroxide, calcium carbonate, magnesium hydroxide, calcium hydroxide, and ammonia may be used. In the present invention, as the basic solution, sodium hydroxide (NaOH) may be used. Conventionally, decellularization was performed using a surfactant and an enzyme. However, in this case, the finally produced extracellular matrix scaffold had problems of not maintaining porosity in its structure and inhibiting cell growth. In the present invention, since the basic solution is used in the decellularization step, there is an advantage of having no cytotoxicity.

In one embodiment, the concentration of the basic solution may be 0.01 to 1 N, 0.06 to 0.45 N, 0.06 to 0.2 N, or 0.08 to 1.02 N. In the above concentration range, an extracellular matrix scaffold having a structure from which cells are easily removed and which has interconnected pores and does not collapse may be produced.

In addition, in one embodiment, the decellularization step may be performed for 60 to 48 minutes, 70 to 200 minutes, or 90 to 150 minutes. In the above time range, an extracellular matrix scaffold having a structure from which cells are easily removed and which has interconnected pores and does not collapse may be produced.

In the present invention, after the decellularization step, a centrifugation step may be further performed before the lyophilization step. Through the centrifugation step, impurities generated in the delipidation step and the decellularization step may be removed, and a high purity extracellular matrix material (precipitate) may be obtained.

In one embodiment, the centrifugation may be performed at 4,000 to 10,000 rpm, or 8,000 rpm for 5 to 30 minutes, 5 to 20 minutes, or 10 minutes.

In addition, before and/or after centrifugation, a washing step may be additionally performed, and for washing, sterile distilled water may be used.

In the present invention, the lyophilization step is a step of lyophilizing the product obtained after the above-described step, that is, the decellularization or centrifugation step. The lyophilization is a method of rapidly cooling the tissue that is in a frozen state and absorbing moisture under a vacuum, and through the lyophilization, the moisture in the extracellular matrix material may be adjusted, and an extracellular matrix scaffold having a porous structure having interconnected pores may be produced.

In one embodiment, the lyophilization may be performed at −50 to −80 ° C. for 24 to 96 hours.

In one embodiment, the water content of the lyophilized extracellular matrix scaffold may be 10% or less, or 1 to 8%.

In the present invention, after the lyophilization step, a sterilization step of sterilizing the extracellular matrix scaffold may be further performed. Through the sterilization step, the immunity of the extracellular matrix scaffold may be eliminated, and bacteria may be effectively destroyed.

In one embodiment, the sterilization step may be performed by irradiation, and the irradiation range may be 10 to30 kGy.

In addition, the present invention provides a method of producing an adipose tissue-derived extracellular matrix scaffold, which includes: a washing step of washing adipose tissue;

a delipidation step of removing a lipid component from the washed adipose tissue;

a decellularization step of removing cells from the lipid component-removed adipose tissue;

a centrifugation step of centrifuging the decellularized adipose tissue;

a lyophilization step of lyophilizing a precipitate after the centrifugation; and

a sterilization step of sterilizing the dried product.

The above steps may be carried out as described above.

In addition, the present invention provides an adipose tissue-derived extracellular matrix scaffold produced by the above-described method of producing an adipose tissue-derived extracellular matrix scaffold.

In one embodiment, the extracellular matrix scaffold may have a moisture content of 10% or less.

In addition, in one embodiment, the extracellular matrix scaffold may have a pore size of 10 to 800 μm, or 100 to 500 μm, and a porosity of 30 to 80%, or 40 to 60%.

The extracellular matrix scaffold may have a porous structure having components similar to the human body, a wide surface area, and interconnected pores. Accordingly, the extracellular matrix scaffold of the present invention may have high cell affinity, and allow cells to survive for a long time. Therefore, the extracellular matrix scaffold may be used as a support for the replacement and reinforcement of damaged human tissue and cell culture.

The present invention will be described in further detail regarding the following examples. However, the scope of the present invention is not limited to the following examples, and it will be understood by those of ordinary skill in the art that various modifications, alterations, or applications are possible without departing from the technical details derived from the details described in the accompanying claims.

### EXAMPLES

### Example 1. Production of Human Adipose Tissue-Derived Extracellular Matrix Scaffold

Fat was eliminated by pulverizing human adipose tissue using a grinder. To remove the fat that was not eliminated, delipidation was carried out using 40% to 60% isopropyl alcohol and 40% to 60% hexane for 16 hours. Cells were removed by treating the fat-removed tissue with 0.01 to 1N sodium hydroxide (NaOH).

To wash the fat- and cell-removed extracellular matrix, supernatant was removed by centrifugation at 8,000 rpm for 10 minutes, and the washing procedure was repeated 5 to 10 times. The scaffold was lyophilized such that the water content in the human adipose tissue-derived extracellular matrix was 10% or less, preferably 1% to 8%, and sterilized by irradiation, thereby producing an extracellular matrix scaffold.

Table 1 shows the result of observing the change in the produced extracellular matrix scaffold over treatment time after adipose tissue was immersed in various concentrations of sodium hydroxide during decellularization.

TABLE 1

| Time | Concentration (N) | | | | |
|---|---|---|---|---|---|
| (Hrs) | 0.01 | 0.05 | 0.1 | 0.5 | 1 |
| 1 | Cell | Cell | Cell | Cell | Structure |
| | removal (x) | removal (x) | removal (x) | removal (x) | collapsed |
| 2 | Cell | Cell | O | Structure | Structure |
| | removal (x) | removal (x) | | collapsed | collapsed |
| 4 | Cell | Cell | Structure | Structure | Structure |
| | removal (x) | removal (x) | collapsed | collapsed | collapsed |
| | | Structure | | | |
| | | collapsed | | | |
| 8 | Cell | Cell | Structure | Structure | Structure |
| | removal (x) | removal (x) | collapsed | collapsed | collapsed |
| | | Structure | | | |
| | | collapsed | | | |
| 16 | Cell | Cell | Structure | Structure | Structure |
| | removal (x) | removal (x) | collapsed | collapsed | collapsed |
| | Structure | Structure | | | |
| | collapsed | collapsed | | | |

From the results shown in Table 1, the optimal concentration (0.1N) and time (2 hrs) for sodium hydroxide treatment can be confirmed. At the above-mentioned concentration and time, an extracellular matrix scaffold having a structure in which cells are easily removed and pores are interconnected, and which does not collapse can be produced.

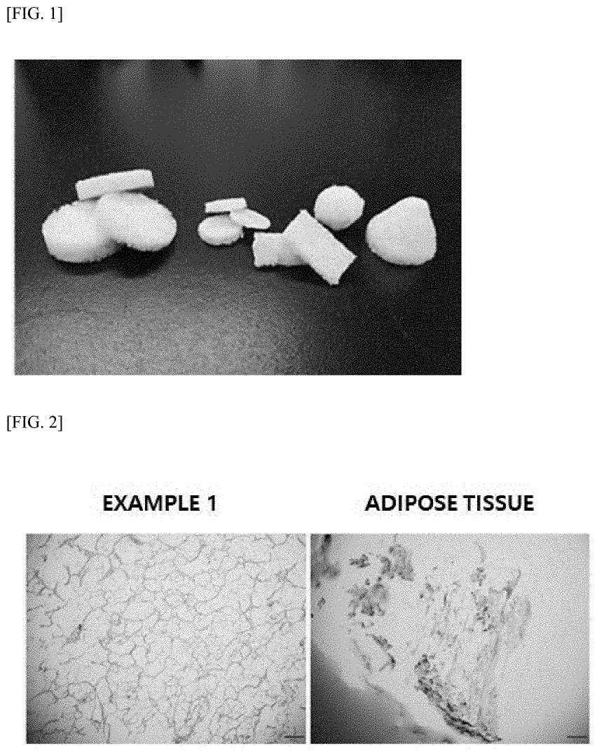

In addition, FIG. 1 is an image of the scaffold produced in Example 1.

As shown in FIG. 1, it can be confirmed that the extracellular matrix scaffold produced by the production method of the present invention has a large surface area and an interconnected porous structure.

### Experimental Example 1. Confirmation of Residual Fat of Human Adipose Tissue-Derived Extracellular Matrix

(1) Method

The human adipose tissue-derived extracellular matrix scaffold produced by the method shown in Example 1 was used as an experimental group, and adipose tissue was used as a control.

To evaluate the residual fat of the extracellular matrix scaffold, Oil Red O staining was performed.

(2) Results

The results of the Oil Red O staining were shown in FIG. 2.

As shown in FIG. 2, it can be confirmed that fat is removed from the human adipose tissue-derived extracellular matrix scaffold produced by the method described in Example 1.

### Experimental Example 2. Confirmation of Residual Cells of Human Adipose Tissue-Derived Extracellular Scaffold

(1) Method

The human adipose tissue-derived extracellular matrix scaffold prepared by the method of Example was used as an experimental example, and adipose tissue was used as a control.

To qualitatively evaluate the residual cells, DAPI staining was performed. In addition, to quantitatively evaluate the residual cells, DNA content was measured.

(2) Results

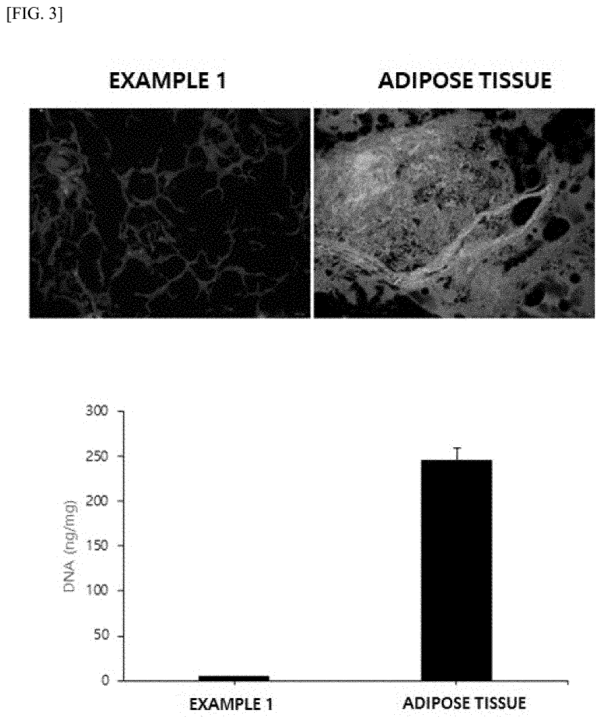

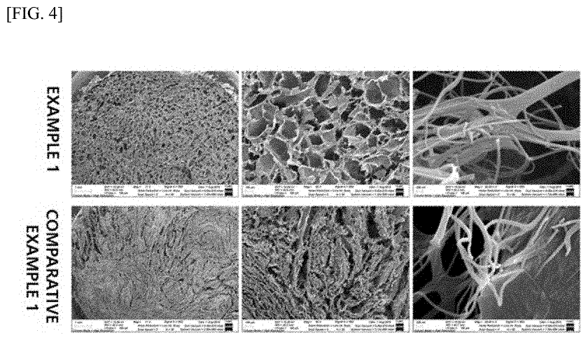

The result of measuring the residual cells is shown in FIG. 3.

FIG. 3A shows a set of images stained by DAPI staining and FIG. 3B is a graph quantifying DNA content.

As shown in FIG. 3, it can be confirmed that cells are removed from the human adipose tissue-derived extracellular matrix scaffold produced by the method of Example 1, and in addition, the content of DNA extracted from the extracellular matrix scaffold is 50 ng/mg or less.

### Comparative Example 1

A human adipose tissue-derived extracellular matrix scaffold was produced by a conventional method (a surfactant and an enzyme).

First, human adipose tissue was washed for 2 days. To further wash fat, the fat was treated with 0.5N NaCl for 4 hours, and 1N NaCl for 4 hours. After washing, the adipose tissue was treated with 0.25% trypsin (enzyme) and EDTA for 2 hours.

The enzyme-treated adipose tissue was treated with 100% isopropyl alcohol for 16 hours for delipidation. To further remove cells, the resulting tissue was treated with 1% trypsin for 3 days.

The extracellular matrix from which fat and cells were removed was washed for 2 days. The scaffold was lyophilized so that the moisture content in the human adipose tissue-derived extracellular matrix is 10% or less, preferably, 1% to 8%, and sterilized by irradiation.

### Experimental Example 3. Confirmation of function of human adipose tissue-derived extracellular scaffold

3-1. Scanning Electron Microscopy of Human Adipose Tissue-Derived Extracellular Matrix Scaffold

(1) Method

The human adipose tissue-derived extracellular matrix scaffold produced by the method of Example 1 was used as an experimental group, and the human adipose tissue-derived extracellular matrix scaffold produced by the method of Comparative Example 2 was used as a control.

Through scanning electron microscopy, the porous structures of the extracellular matrix scaffolds of Example 1 and Comparative Example 1 were analyzed.

(2) Results

The result of analyzing the porous structures is shown in FIG. 4. FIG. 4 shows a set of images photographed by a scanning electron microscope.

As shown in FIG. 4, it can be qualitatively confirmed that, in the extracellular matrix scaffold produced in Comparative Example 1, that is, the control, porosity is not uniform and its structure has collapsed, but the human adipose tissue-derived extracellular matrix scaffold produced by the method of Example 1 has a pore structure which has uniform porosity and connected pores without the structure collapsing.

2-2. Confirmation of Cell Growth in Human Adipose Tissue-Derived Extracellular Matrix Scaffold

(1) Method

An experiment for cell growth in the extracellular matrix scaffold was performed using the human adipose tissue-derived extracellular matrix scaffold prepared by the method of Example 1 as an experimental group, and the human adipose tissue-derived extracellular matrix scaffold prepared by the method of Comparative Example 1 as a control.

1×10⁵ cells/100 μl of fibroblasts were seeded on the scaffold and immersed in a culture medium to perform a culture.

To evaluate cell growth, on days 1, 7, and 14 after the culture, the cells were stained with a live/dead cell viability assay kit (Life Technology, USA). The scaffold was immersed in the medium in which 0.5 μl/ml calcein-AM and 2 μl /ml ethidium homodimer-1 are dissolved to allow a reaction for 30 minutes. After the reaction, the scaffold was confirmed using a confocal microscope (LSM 700, Carl Zeiss, Germany). Cell survival in the scaffold was confirmed by focusing to a depth of approximately 200 μm and at an interval of 10 μm.

(2) Results

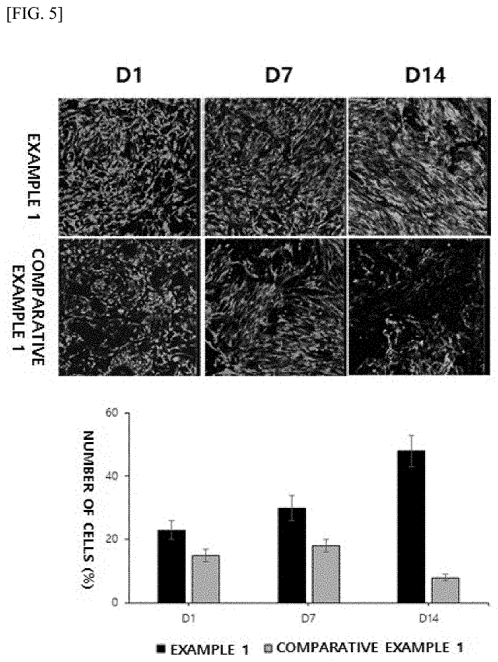

The result of confirming cell growth is shown in FIG. 5.

FIG. 5 shows a set of images obtained using a live/dead cell viability assay kit and a quantitative graph to analyze cell growth.

As shown in FIG. 5, it can be confirmed that, in the human adipose tissue-derived extracellular matrix scaffold produced by the method of Example 1, the number of living cells increases over time, compared to Comparative Example 1, that is, the control. In addition, from the graph, it can be confirmed that the number of cells increases 5-fold compared to the control on day 14 after the culture.

### INDUSTRIAL APPLICABILITY

The extracellular matrix scaffold may have a porous structure having components similar to the human body, a wide surface area, and interconnected pores. Accordingly, the extracellular matrix scaffold of the present invention may have high cell affinity, and allow cells to survive for a long time. Therefore, the extracellular matrix scaffold may be used as a support for the replacement and reinforcement of damaged human tissue and cell culture.

1. A method of producing an adipose tissue-derived extracellular matrix scaffold, comprising:
a delipidation step of removing a lipid component from adipose tissue;
a decellularization step of removing cells from the lipid component-removed adipose tissue; and
a lyophilization step of lyophilizing the cell-removed adipose tissue,
wherein the decellularization step is performed using a basic solution.

2. The method of claim 1, wherein the adipose tissue is allogeneic or heterologous adipose tissue.

3. The method of claim 1, wherein the removal of the lipid component is performed by physical treatment and/or chemical treatment.

4. The method of claim 3, wherein the physical treatment is pulverization.

5. The method of claim 3, wherein the chemical treatment is performed using a delipidation solution, and
the delipidation solution is a polar solvent, a non-polar solvent, or a mixed solvent thereof.

6. The method of claim 1, wherein the basic solution comprises one or more selected from the group consisting of sodium hydroxide, potassium hydroxide, ammonium hydroxide, calcium carbonate, magnesium hydroxide, calcium hydroxide, and ammonia.

7. The method of claim 1, wherein the concentration of the basic solution is 0.01 to 0.1 N, and
the treatment time is 60 to 480 minutes.

8. The method of claim 1, further comprising a centrifugation step after the decellularization step.

9. The method of claim 1, wherein the lyophilization step is performed at −50 to −80 ° C. for 24 to 96 hours.

10. The method of claim 1, further comprising a sterilization step after the lyophilization step.

11. An adipose tissue-derived extracellular matrix scaffold that is produced by the production method of claim 1, and has a moisture content of 10% or less.
